(19) United States
(12) Patent Application Publication
(10) Pub. No.: US 2015/0272881 A1
(43) Pub. Date: Oct. 1, 2015

(54) REDUCED PYRROLOQUINOLINE QUINONE GEL

(71) Applicant: MITSUBISHI GAS CHEMICAL COMPANY, INC., Tokyo (JP)

(72) Inventor: Kazuto Ikemoto, Niigata-shi (JP)

(21) Appl. No.: 13/261,856

(22) PCT Filed: Nov. 15, 2012
(86) PCT No.: PCT/JP2012/079709

§ 371 Date: Jul. 15, 2014

(30) Foreign Application Priority Data
Nov. 15, 2011 (JP) 2011-249-341 2011

(51) Int. Cl. A61K 9/06; A23L 1/30; A61K 8/04; A61K 31/4745; A61K 8/49

(52) U.S. Cl. CPC A61K 9/06; A61K 31/4745; A61K 8/4926; A61K 8/042; A23L 1/30; A23V 2002/00

(57) ABSTRACT

An object of the present invention is to provide a gel comprising a reduced pyrroloquinoline quinone derivative or a salt thereof and a method for producing the same in a simple way and with high efficiency. According to the present invention, there is provided a gel comprising a reduced pyrroloquinoline quinone derivative or a salt thereof by mixing an oxidized pyrroloquinoline quinone derivative or a salt thereof, a dispersant, and a reducing agent at 50° C. or lower, and a method for producing the same.

### CROSS-REFERENCE TO RELATED APPLICATION

This application enjoys the benefit of Japanese Patent Application No. 2011-249341, filed on Nov. 15, 2011. The disclosure of this earlier application is incorporated herein by reference in its entirety.

### TECHNICAL FIELD

This invention relates to a gel containing reduced pyrroloquinoline quinone and a method for producing the gel.

### BACKGROUND ART

Oxidized pyrroloquinoline quinone is generally referred to as PQQ, and has been proposed as a possible novel vitamin. There is much attention to it as a useful substance for dietary supplements, cosmetics, and the like. Also, PQQ has been found to have many physiological activities such as cell growth-promoting activity, anti-cataract activity, hepatic disease-preventing and therapeutic activity, wound healing activity, antiallergic activity, reverse transcriptase-inhibiting activity, glyoxalase I-inhibiting activity-anticancer activity, and the like.

It has been reported that reduced PQQ has higher scavenging performance of reactive oxygen compared to oxidized PQQ (Non-patent document 4), and there is a demand for its provision. Reduced PQQ has been reported to be produced from oxidized PQQ by reacting it with a reducing agent (Non-patent document 5), and there is a report describing that reduced PQQ can be produced based on the redox potential of ascorbic acid (Patent document 1), however, reduced PQQ was not actually isolated.

As just described, there have been many reports about oxidized or reduced PQQ. However, its properties as a substance are not well known. Only the crystal structure of PQQ disodium has been reported (Non-patent document 1).

On the other hand, gel-like substances have been applied not only to foods, medicines, cosmetics, and chromatography but also to familiar athletic shoes, deodorants, and the like. Since gel-like substances are palatable and easy in handling, unlike liquid, they are easily swallowed. Therefore, the use of gel-like substances is particularly desired for those with impaired swallowing function (difficulty in swallowing food) due to aging or diseases and those who find it difficult to swallow hard tablets.

In addition, coating technology is important in the fields of foods and medicines, and there is a demand to form a uniform coating layer. However, it is known to be difficult to form a uniform coating layer on usual crystals, but easy on crystals having a long configuration (Non-patent document 6). From this viewpoint, a gel has a fibrous configuration, and thus is suitable for this purpose.

So far, gellants used in the food and pharmaceutical fields are macromolecular substances represented by collagen, hyaluronic acid, agar and carrageenan. Besides such macromolecular compounds, some low-molecular weight compounds are reported to form a gel as gellants (Non-patent documents 2 and 3). However, low-molecular weight gellants are known to be used for food products. Furthermore, gellants for use in the food field are required to form a gel at around room temperature for preventing denaturation, although many of the macromolecular gellants are often formed by dissolution under heating followed by cooling.

### PRIOR ART DOCUMENT

### Patent Document

Patent document 1: EP 0656791

### Non-Patent Document

Non-patent document 1: JACS, vol. 111, 6822-6828 (1989)
Non-patent document 2: JACS, vol. 122, 11679-11691 (2000)
Non-patent document 3: Angew. Chem. Int. Ed. vol. 39, 3447-3450 (2000)
Non-patent document 4: K. Mukai, A. Ouchi, M. Nakano, J. Agric. Food Chem. 2011, 59, 1705.
Non-patent document 5: J. A. Duine et al. Eur. J. Biochem. 118, 395-399 (1981)
Non-patent document 6: P. J. Yunker, T. Still, M. A. Lohr, A. G. Yodh, Nature, 467, 308-311, 2011

### SUMMARY OF THE INVENTION

### Problem to be Solved by the Invention

The inventors have found that a gel comprising a salt of reduced pyrroloquinoline quinone formed into a fibrous structure is obtained by mixing an aqueous solution of the salt of oxidized pyrroloquinoline quinone and a reducing agent at room temperature (Example 1). According to the present invention, the salt of reduced pyrroloquinoline quinone can form a gel without being dissolved in a dispersant (solvent) under predetermined conditions. The present invention is based on this finding.

An object of the present invention is to provide a reduced pyrroloquinoline quinone gel and a method for producing thereof in a simple way and with high efficiency.

### Means for Solving Problem

According to the present invention, the following inventions are provided:

(1) A gel comprising a reduced pyrroloquinoline quinone derivative represented by Formula (1) or a salt thereof:

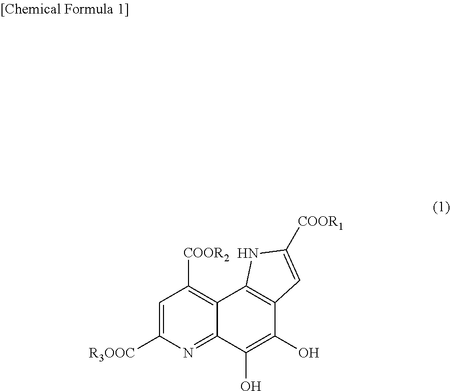

wherein R₁, R₂, and R₃, which may be the same or different, represent a hydrogen atom, a phenyl group, or an alkyl group having 1 to 6 carbon atoms, an aralkyl group, an alkylaryl group, an alkenyl group or an alkynyl group, and a dispersant.

(2) The gel of (1), wherein the reduced pyrroloquinoline quinone derivatives or salts thereof are associated with each other to form a fibrous structure.
(3) The gel of (1) or (2), wherein the reduced pyrroloquinoline quinone derivative or a salt thereof is present in an amount of 0.001 to 70% by weight based on the total weight of the gel.
(4) The gel according to (1) or (2), wherein the reduced pyrroloquinoline quinone derivative or a salt thereof is present in an amount of 0.05% by weight or more and less than 0.7% by weight based on the total weight of the gel.
(5) The gel according to any one of (1) to (4), further comprising a reducing agent.
(6) The gel according to any one of (1) to (5), wherein the dispersant is water.
(7) The gel according to any one of (1) to (6), further comprising an oxidized pyrroloquinoline quinone derivative or a salt thereof represented by Formula (2):

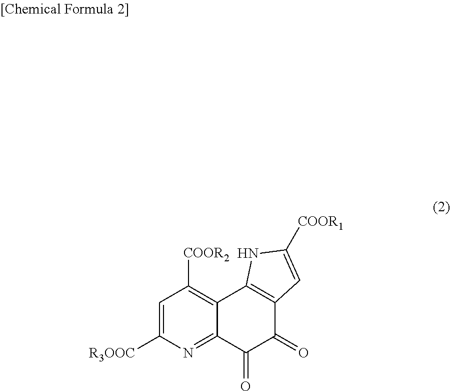

wherein R₁, R₂, and R₃, which may be the same or different, represent a hydrogen atom, a phenyl group, or an alkyl group having 1 to 6 carbon atoms, an aralkyl group, an alkylaryl group, an alkenyl group or an alkynyl group.

(8) The gel according to (7), wherein the reduced pyrroloquinoline quinone derivative or a salt thereof is contained in an amount of 0.1 mol % or more in the total molar number of the pyrroloquinoline quinone derivative or a salt thereof.
(9) The gel according to any one of (1) to (8), further comprising a macromolecular gellant.
(10) A dry product which is obtained by drying the gel according to any one of (1) to (9).
(11) A film produced from the gel according to any one of (1) to (9).
(12) A food product comprising the gel of any one of (1) to (9) or the dry product of (10).
(13) A pharmaceutical product comprising the gel of any one of (1) to (9) or the dry product of (10).
(14) A cosmetic product comprising the gel of any one of (1) to (9) or the dry product of (10).
(15) A method for producing a gel comprising a reduced pyrroloquinoline quinone derivative or the salt thereof represented by Formula (1),

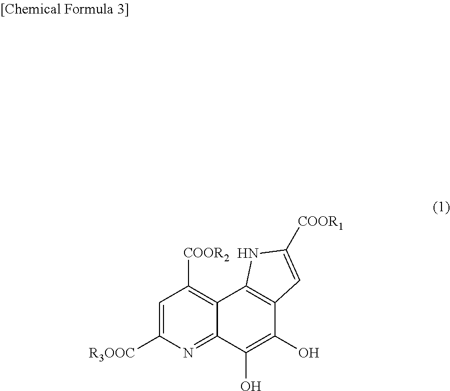

wherein R₁, R₂, and R₃, which may be the same or different, represent a hydrogen atom, a phenyl group, or an alkyl group having 1 to 6 carbon atoms, an aralkyl group, an alkylaryl group, an alkenyl group or an alkynyl group, said method comprising mixing an oxidized pyrroloquinoline quinone derivative or a salt thereof represented by Formula (2):

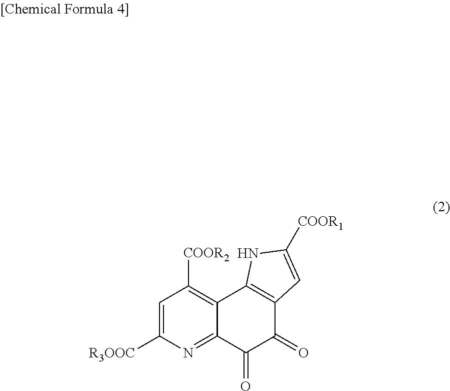

wherein R₁, R₂, and R₃, which may be the same or different, represent a hydrogen atom, a phenyl group, or an alkyl having 1 to 6 carbon atoms, an aralkyl group, an alkylaryl group, an alkenyl group, or an alkynyl group,

a dispersant, and a reducing agent at 50° C. or lower.
(16) The production method according to (15), wherein the concentration of the oxidized pyrroloquinoline quinone derivative or a salt thereof in the dispersant is 0.05% by weight or more and less than 0.7% by weight.

### Effect of the Invention

The present invention provides a gel containing a reduced PQQ derivative or a salt thereof and a dispersant such as water and the like. In the present invention, the reduced PQQ derivative or the salt thereof can produce a low molecular gel at a low concentration, and the gel is edible.

The gel of the present invention can produce a uniform coating, and the gel is also provided as a raw material for a film and the like.

The gel of the present invention can be produced even without the use of conventional gellants. In addition, gelation can be carried out in a simple way without a need of treatment at high temperature that is required for conventional gelation.

### BRIEF DESCRIPTION OF THE DRAWINGS

FIG. 1 shows a photograph of a gel of Example 5.

FIG. 2 shows an optical micrograph of a gel of Example 5.

FIG. 3 shows a UV spectrum of the sample 1, in Example 6.

FIG. 4 shows a UV spectrum of the sample 2, in Example 6.

FIG. 5 shows a UV spectrum of the sample 3, in Example 6.

FIG. 6 shows a UV spectrum of the sample 4, in Example 6.

FIG. 7 shows the results of X-ray diffraction of a dried gel.

FIG. 8 shows an optical micrograph of a gel in agar.

FIG. 9 shows an optical micrograph of a gel of Example 1.

### EMBODIMENTS FOR CARRYING OUT THE INVENTION

According to the present invention, a gel comprising a reduced pyrroloquinoline quinone derivative or a salt thereof can be produced by mixing an oxidized pyrroloquinoline quinone derivative or a salt thereof, a dispersant, and a reducing agent at 50° C. or lower.

The term “a gel comprising a reduced pyrroloquinoline quinone derivative or a salt thereof” means a gel substantially consisting of the reduced pyrroloquinoline quinone derivative or a salt thereof and a dispersant. In the gel of the present invention, the reduced pyrroloquinoline quinone derivatives or salts thereof are associated with each other to form a fibrous structure, and the dispersant is incorporated in the fibrous structure.

The “fibrous structure” as used herein refers to a three-dimensional network structure formed by self-association of the pyrroloquinoline quinone derivative or a salt thereof via interactions other than a covalent bond to form an association thereof and a physically cross-linking the association. That is, the gel of the present invention may be called a physical gel. The interactions other than a covalent bond include non-covalent bonds such as a hydrogen bond, an ionic bond, a coordination bond, π-π interactions (stacking), hydrophobic interactions and the like, and, in particular, a hydrogen bond and an ionic bond.

The gel of the present invention substantially consists of the reduced PQQ derivative or a salt thereof represented by the following formula (1) and the dispersant. The gel of the present invention is a gel in which a fibrous structure is formed by physically cross-linking from the reduced PQQ derivative or a salt thereof.

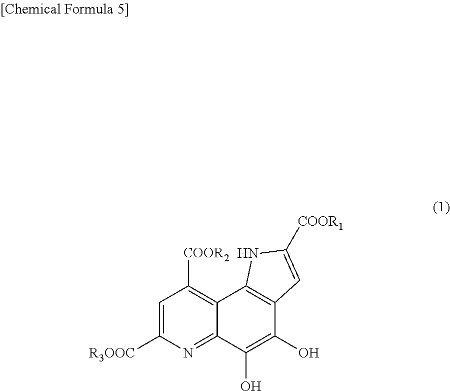

wherein, R₁, R₂, and R₃, which may be the same or different, represent a hydrogen atom, a phenyl group, or an alkyl group having 1 to 6 carbon atoms, an aralkyl group, an alkylaryl group, an alkenyl group or an alkynyl group.

The gel of the present invention optionally comprises an oxidized PQQ derivative or a salt thereof represented by Formula (2):

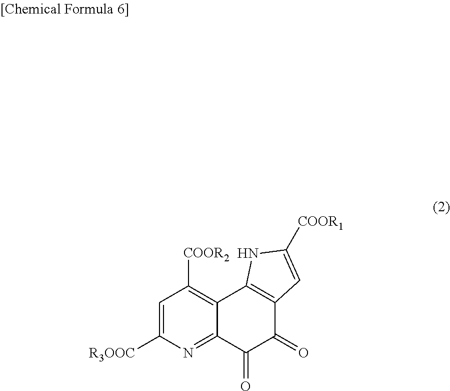

wherein, R₁, R₂, and R₃, which may be the same or different, represent a hydrogen atom, a phenyl group, or an alkyl group having 1 to 6 carbon atoms, an aralkyl group, an alkylaryl group, an alkenyl group or an alkynyl group.

In Formulae (1) and (2), the term “an alkyl group” means a linear or branched alkyl group. Examples of “an alkyl group having 1 to 6 carbon atoms” include methyl, ethyl, n-propyl, i-propyl, n-butyl, i-butyl, s-butyl, t-butyl, n-pentyl, neopentyl, i-pentyl, t-pentyl, n-hexyl, i-hexyl, and the like.

The term “an aralkyl group” in Formulae (1) and (2) means an alkyl group wherein one of the hydrogen atoms on the alkyl group is substituted with an aryl group. Preferably, it is an aralkyl group having 7 to 12 carbon atoms. Examples of “an aralkyl group” include a benzyl group, a phenethyl group, a phenylpropyl group, a phenylbutyl group, and the like.

The term “an alkylaryl group” in Formulae (1) and (2) means an aryl group wherein one of the hydrogen atoms on the aryl group is substituted with an alkyl group. Preferably, it is an alkylaryl group having 7 to 12 carbon atoms. Examples of “an aralkyl group” include a benzyl group, a phenethyl group, a phenylpropyl group, a phenylbutyl group, and the like.

The term “an alkenyl group” in Formulae (1) and (2) means a linear or branched alkenyl group. Preferably, it is an alkenyl group having 2 to 6 carbon atoms. Examples of “an alkenyl group” include a vinyl group, an allyl group, a 1-propenyl group, an isopropenyl group, a 1-buten-1-yl group, a 1-buten-2-yl group, a 1-buten-3-yl group, a 2-buten-1-yl group, a 2-buten-2-yl group, and the like.

The term “an alkynyl group” in Formulae (1) and (2) means a linear or branched alkynyl group. Preferably, it is an alkynyl group having 2 to 6 carbon atoms. Examples of “an alkynyl group” include an ethynyl group, a 1-propynyl group, a 2-propynyl group, a butynyl group, a pentynyl group, a hexynyl group, and the like.

Preferably, R₁ is a hydrogen atom or an alkyl group having 1 to 6 carbon atoms, more preferably a hydrogen atom or a methyl group, and further preferably a hydrogen atom.

Preferably, R₂ is a hydrogen atom or an alkyl group having 1 to 6 carbon atoms, more preferably a hydrogen atom or a methyl group, and further preferably a hydrogen atom.

Preferably, R₃ is a hydrogen atom or an alkyl group having 1 to 6 carbon atoms, more preferably a hydrogen atom or a methyl group, and further preferably a hydrogen atom.

The reduced pyrroloquinoline quinone derivative represented by Formula (1) is a compound wherein any of R₁, R₂, and R₃ is preferably a hydrogen atom or an alkyl group having 1 to 6 carbon atom(s), more preferably a hydrogen atom or a methyl group, and further preferably a hydrogen atom (so called a reduced pyrroloquinoline quinone).

The oxidized pyrroloquinoline quinone derivative represented by Formula (2) is a compound wherein any of R₁, R₂, and R₃ is preferably a hydrogen atom or a alkyl group having 1 to 6 carbon atoms, more preferably is a hydrogen atom or a methyl group, and further preferably a hydrogen atom (so called pyrroloquinoline quinone).

Salts of the reduced PQQ derivative of Formula (1) or oxidized PQQ derivative of Formula (2) used in the present invention (hereinafter, when “a reduced pyrroloquinoline quinone derivative of Formula (1)” and “an oxidized pyrroloquinoline quinone derivative of Formula (2)” are not discriminated, they are referred to as “a pyrroloquinoline quinone derivative” or “a PQQ derivative”) include an alkali-metal salt, an alkali-earth metal salt, an ammonium salt, and the like. In particular, the alkali-metal salt is preferable because it is soluble in water.

The alkali metal salt of pyrroloquinoline quinone used in the present invention includes a salt of sodium, potassium, lithium, calcium, magnesium, cesium, rubidium, and the like. Preferred is the sodium salt.

The alkali metal salt of PQQ derivative may be substituted with one to three atoms of alkali metals to form an alkali metal salt thereof, which may be any of a monoalkali metal salt, a dialkali metal salt and a trialkali metal salt, preferably a dialkali metal salt. The alkali metal salt of PQQ derivative is especially preferably the disodium salt.

The oxidized PQQ derivative of Formula (2) or a salt thereof can be commercially available, and can also be produced by publicly known methods.

The oxidized pyrroloquinoline quinone derivative or a salt thereof is preferably a salt of the oxidized pyrroloquinoline quinone derivative.

The reduced pyrroloquinoline quinone derivative or a salt thereof is preferably a salt of the reduced pyrroloquinoline quinone derivative.

The reduced PQQ derivative or a salt thereof is preferably contained in an amount of 0.1 to 100 mol % with respect to the total PQQ (both the reduced PQQ and oxidized PQQ) derivatives or a salt thereof that are contained in the gel. More preferably, the content of the reduced PQQ derivative or a salt thereof is 50 mol % or more, further preferably the content of the reduced PQQ derivative or a salt thereof is 60 mol % or more, further preferably the content of the reduced PQQ derivative or a salt thereof is 70 mol % or more, in particular preferably the content of the reduced PQQ derivative or a salt thereof is 80 mol % or more, and the most preferably the content of the reduced PQQ derivative or a salt thereof is 90 mol % or more. The reduced PQQ derivative or a salt thereof is less easily dissolved compared to the oxidized PQQ derivative or a salt thereof, and tends to form a gel structure, therefore, the reduced PQQ derivative or a salt thereof preferably is present as much as possible within the above range.

The gel of the present invention preferably comprises the reduced PQQ derivative or a salt thereof in an amount of 0.001 to 70% by weight in the total weight of the gel, more preferably 0.05 to 5% by weight, further preferably 0.05% by weight or more but less than 0.7% by weight, further more preferably 0.05 to 0.4% by weight, and in particular preferably 0.05 to 0.3% by weight. When the concentration is lower than these, it is dissolved in solvent such as purified water and the like, resulting in no gelation. When the concentration is higher than this range, it turns to be a clay-like substance that makes it difficult to assess whether gel configuration is formed.

In the gel of the present invention, the configuration of the reduced PQQ derivative or a salt thereof or oxidized PQQ derivative or a salt thereof is in a form of a solid state, regardless of amorphous or crystalline form. Crystallization is considered to progress from the amorphous state. Crystallization can be assessed by X-ray diffraction. For example, when X-ray diffraction was performed using an equipment: RINT2500 manufactured by RIGAKU Corporation, X-ray source: Cu/tube voltage of 40 kV/tube current of 100 mA, scan rate: 4.000°/min, sampling width: 0.020°, the reduced PQQ derivative or a salt thereof in the gel of the present invention is a crystalline material having diffraction peaks at 8.14, 10.41, 19.74, and 29.94±0.08°.

A gel having a fibrous structure can be produced by mixing an oxidized pyrroloquinoline quinone derivative or a salt thereof, a dispersant, and a reducing agent at 50° C. or lower through the association of the reduced pyrroloquinoline quinone derivative or a salt thereof with each other.

The mixing of the oxidized pyrroloquinoline quinone derivative or a salt thereof, the dispersant, and the reducing agent can be carried out by mixing each ingredient simultaneously or separately. When each ingredient is mixed separately, the ingredients may be mixed in any order. Preferably, first the oxidized pyrroloquinoline quinone derivative or a salt thereof and the dispersant are mixed, and the resultant mixture (solution) can be mixed with the reducing agent. In this step, the reducing agent can be also mixed with the dispersant.

Examples of the dispersant include water, organic solvent, oil and fat, and the like. Examples of the organic solvent include ethanol, propanol, butanol, glycerin, propylene glycol, ethyl lactate, and methyl α-hydroxyisobutyrate. Examples of the oil and fat include rice-bran oil, coconut oil, corn oil, olive oil, rapeseed oil, triacetin, soybean oil, medium-chain glycerin ester, and the like. The dispersant is preferably water. When the dispersant is water, hydrated gel (hydrogel) can be prepared. The dispersant can be possibly exchanged.

A reducing agent that can be used is not particularly limited, and sodium borohydride, Na₂S₂O₄, phenylhydrazine, hydrogen, phenylthiol, NADPH, NADH, ascorbic acid, glutathione, cysteine, and the like can be used as a typical reducing agent. Hydrogen, NADPH, NADH, ascorbic acid, glutathione, and cysteine that are highly safe are preferred. Ascorbic acid is more preferred.

Examples of as ascorbic acid also include compounds similar to ascorbic acid (ascorbic acid analogues), for example rhamno-ascorbic acid, arabo-ascorbic acid, gluco-ascorbic acid, fuco-ascorbic acid, glucohepto-ascorbic acid, xylo-ascorbic acid, galacto-ascorbic acid, gulo-ascorbic acid, allo-ascorbic acid, erythro-ascorbic acid, 6-desoxyascorbic acid and the like. It may be also be an ester or a salt (for example, palmitate, stearate, sodium salts, calcium salts, etc.). Further, these may be L-isomer (for example, L-ascorbic acid, sodium L-ascorbate, and the like), D-Isomer (for example, D-arabo-ascorbic acid, sodium D-arabo-ascorbate, and the like), or a racemic mixture (racemate).

The reducing agent used in the present invention can be used either as a reducing agent itself or a reducing agent solution. When the reducing agent is used as a solution, it can be used by dissolving in the dispersant, however, an aqueous solution of the reducing agent is preferably used.

The reducing agent solution can be prepared, for example, at a concentration of 0.1 to 500 g/L, and preferably 0.5 to 150 g/L.

The amount of the reducing agent used may be adjusted to 0.1 to 5000-fold molar number with respect to the oxidized PQQ derivative or a salt thereof. Since the addition of an excess amount of the reducing agent facilitates the reaction, 1.1 to 1000-fold is preferred, and more preferably, 1.1 to 100-fold. That is, in the production method of the present invention, the molar ratio between the oxidized PQQ derivative or a salt thereof and the reducing agent can be adjusted by 1:0.1 to 5000, preferably, 1:1.1 to 1000, and more preferably 1:1.1 to 100. The reducing agent may remain as a residual inside the gel, and the excess amount of the reducing agent can strengthen the maintenance of the reduced PQQ derivative or a salt thereof, enabling the production of stable products.

The method for producing the gel of the present invention may vary depending on the conditions such as the concentration of salts, precursors, temperature, and the like; however, it can be generalized as follows. The gelation is made possible by adding the oxidized PQQ or a salt thereof to a dispersant and subjecting the resultant mixture to a reaction by further adding a reducing agent at 50° C. or lower at pH 0 to 14.

Oxidized PQQ may be completely dissolved in the dispersant, or may remain as residual in a suspension.

The oxidized PQQ derivative or a salt thereof may be added to make 0.001 to 70% by weight in the dispersant, preferably 0.005 to 10% by weight, and more preferably 0.05% by weight or more and less than 0.7% by weight. High concentration enables the gel to solidify and stabilize, but the amount of oxidized PQQ or reduced PQQ increases, which is not economical. In addition, a part that is not used for forming the gel structure increases, therefore it is not efficient. On the other hand, the concentration lower than that described above tends to make the gel disintegrate.

The concentration of the oxidized pyrroloquinoline quinone derivative or a salt thereof can be less than its solubility. The “solubility” in this description means the limit of dissolving of a solute in a solvent; it can be expressed as the concentration of a solute in a saturated solution. The solubility of the oxidized pyrroloquinoline quinone derivative or a salt thereof can be appropriately decided depending on the temperature of the mixture. For example, the solubility of the oxidized pyrroloquinoline quinone disodium is 0.299 g with respect to 100 g of water at 25° C.

The weight concentration of the oxidized PQQ derivative or a salt thereof can be adjusted at 0.001 to 70% by weight in the dispersant, preferably 0.005 to 10% by weight, more preferably 0.05% by weight or more and less than 0.7% by weight, further preferably 0.05 to 0.4% by weight, and furthermore preferably 0.05 to 0.3% by weight.

The oxidized PQQ derivative or a salt thereof is reduced to a reduced PQQ derivative or a salt thereof by adding the reducing agent, and gelation proceeds through slow growth of a fibrous solid. The reduction reaction and gelation proceed simultaneously. When the temperature is too low, the solubility of oxidized PQQ excessively decreases, resulting in cessation of the reduction reaction. When the temperature is too high, the growth of the fibrous solid does not proceed. Preferably, the reaction temperature is −10° C. to 50° C.

The temperature of the mixture obtained can be adjusted at −10° C. to 50° C. However, it is set preferably at 0 to 45° C., and more preferably at 0 to 30° C. from the view point of operability.

The mixture obtained is preferably reacted for 1 minute or more, and a reaction time of 10 minutes is more preferable because gelation can be achieved without fail. Preferably, the reaction time is 1 min to 72 hours, and more preferably 10 min to 48 hours.

The present inventors have found that a salt of oxidized PQQ is also gelated (WO2012/020767). As described in this patent, when the gel of oxidized PQQ is prepared, high concentration is required, otherwise the salt is dissolved and is not gelated and it is difficult to form a gel from a homogeneous solution. When the gel is formed from oxidized PQQ alone, the concentration at which no complete dissolution occurs is required. To make a stable gel, the concentration is preferably 0.7% by weight or more. At room temperature, the gel is not formed at the concentration of 0.3% by weight.

As the dispersant, examples include water, organic solvent, oil and fat, and the like. Examples of the organic solvent include ethanol, propanol, butanol, glycerin, propylene glycol, ethyl lactate, methyl α-hydroxyisobutyrate, and the like. Examples of the oil and fat include rice-bran oil, coconut oil, corn oil, olive oil, rapeseed oil, triacetin, soybean oil, medium-chain glycerin ester, and the like. The dispersant is preferably water. When the dispersant is water, a hydrated gel (hydrogel) can be produced. In a gelation compound, the dispersant can be exchanged by exchanging the dispersant contained in the gel.

The range of pH may be 0-14, preferably 1-9, and more preferably 1-6. The concentration of the reduced PQQ derivative or a salt thereof is preferably increased since it is easily dissolved under alkaline conditions. In addition, the reduction by ascorbic acid is preferably carried out under acidic conditions.

That is, mixing of the oxidized pyrroloquinoline quinone derivative or a salt thereof with the dispersant and the reducing agent can be performed at pH in a range of 0 to 14, preferably 1 to 9, more preferably 1 to 6, and further preferably 3 to 6.

The state in which the reduced PQQ derivative or a salt thereof and the oxidized PQQ derivative or a salt thereof in the gel are mixed can be prepared by adjusting the ratio of the amount of the oxidized PQQ derivative or a salt thereof and the reducing agent, and the like. Although the presence of the reduced PQQ derivative or a salt thereof strengthens the intermolecular bond and facilitates gelation, it has no particular problem as far as the gel maintains its configuration even though the reduced PQQ derivative or a salt thereof is oxidized to the oxidized PQQ derivative or a salt thereof by oxygen.

According to Examples described below, the present invention is characterized in that PQQ itself can be gelated from the mixture of a salt of PQQ and a dispersant (water), and the gel can be produced without conventional gellants.

The formation of the gel proceeds through spreading of fibrous macromolecular chains over the entire solution, wherein the fibrous chains hold the liquid. The reduced PQQ derivative or a salt thereof is a low molecular compound; therefore, gelation requires that the compounds undergo fibrillation as a macromolecule. In the present invention, it is thought that the formation of the reduced PQQ derivative or a salt thereof makes them insoluble and non-covalent bonding enables the macromolecule formation, resulting in the fibrillation.

As described above, in the gel of the present invention, fibrillation is achieved by intermolecular bond, wherein high temperature is not required in this process as in the conventional method, so the gel can be easily produced.

It is expected that intermolecular interaction of the reduced PQQ derivative or a salt thereof is maintained by an ionic bond with an alkali metal or a hydrogen bond. When the concentration of the PQQ derivative or a salt thereof is high, a substance is generated that has the original crystal structure mixed with the fibrous structure for gelation. However, there is no particular problem as far as gelation is achieved. In addition, there is no problem even though PQQ powder is added to a substance that is once gelated.

When PQQs are to form the fibrous structure, the molecular chain is thought to be extended. The present inventors consider as follows: The crystal structure described in Non-patent document 1 shows that a hydrogen bond and an ionic bond are present between the disodium salts of PQQ, and also aromatic rings are stacked. Although fibrous reduced PQQ has a different structure from its crystal form, this has some relevance to the present situation. Regarding the reduced form, it is thought that the intermolecular bond is enhanced by the involvement of the hydroxyl groups of hydroquinone in forming the hydrogen bond.

When the oxidized pyrroloquinoline quinone derivative or a salt thereof is contained, the association is thought to proceed through the similar interaction.

Although the content of the reduced PQQ derivative or a salt thereof in the resultant gel is determined depending on the initial concentration of the oxidized PQQ derivative or a salt thereof, the concentration can be changed by dilution or concentration. For dilution, a dispersant may be added. Concentration can be performed by removing an excess amount of the dispersant by centrifugation or filtration, or by evaporation of the dispersant. Maintaining the fibrous structure enables formation of a gel containing a solvent.

In the gel of the present invention, the dispersant can be exchanged. For example, when water is used as the dispersant for gelation, water can be exchanged to other dispersant. As a method of exchanging of the dispersant, a method of washing the gel with a dispersant for exchange on a filter, or removing the supernatant by adding a dispersant for exchange repeatedly, is simple. As the dispersant for exchange, substitution with ethanol, propanol, butanol, oil and fat and the like as a liquid has no problem. In the present invention, not only a hydrogel but also a gel containing an organic solvent can be produced.

In order to improve the properties of the gel, commonly used macromolecular gellants can be mixed. Examples of the macromolecular gellant include gelatin, agar, carrageenan, collagen, fucoidan, hyaluronic acid, konnyaku, glucomannan, pectin, Locust bean gum, xanthane gum, gellan gum, starch, egg white, and the like. These agents can be mixed to a gelled state or added simultaneously with gelation.

The gel of the present invention can be used for food products, cosmetic products, pharmaceutical products, pesticides, agricultural supplies, electronic materials, filter materials, and the like.

When the gel is used to produce food products and cosmetic products, other components can be added if necessary. As additives, for example flavoring agent, acidulant, salt, sweetener, umami component, fruit juice, fermented food, lipid, moisturizer, whitening agent, herbal extract, tea, coffee, emulsifier, glycerin, antiseptic agent, anti-microbial agent, steroid, methyl salicylate, vitamin, indometacin, etc. may be added if necessary.

The gel can be mixed with a monosaccharide, a disaccharide, an oligosaccharide, and an artificial sweetener as a sweetener. Examples include fructose, glucose, galactose, sorbitol, xylitol, erythritol, trehalose, Palatinit, aspartame, acesulfame K, sucralose, glycyrrhiza extract, Lo Han Kuo starch syrup, honey, etc.

When a mixture of these ingredients is formed, these ingredients are desirably added to such an extent that the gel of PQQ is not collapsed, and preferably mixed under conditions of room temperature or lower.

For use in food, pharmaceutical, and cosmetic applications, it is natural to be careful from a hygienic point of view, and the production is desirably done in an aseptic environment, for example, clean room.

Fibrous structure (fibrous substance) can be produced from the reduced PQQ derivative or a salt thereof by drying the gel of the present invention. Methods for drying include, for example, lyophilization, spray drying, drying by heating after solvent substitution, and the like. The resultant solid obtained by drying this gel is characterized by a large surface area and fibrous form, which makes it different from usual powder in terms of texture and appearance. Therefore, it is in particular advantageous to the fields of foods, cosmetics and medicines. In addition, casting this on a plate can produce a film-like substance.

The fibrous structure of the present invention can have a fiber thickness of 0.02 to 2000 μm, preferably 0.05 to 500 μm, more preferably 0.05 to 50 μm and further more preferably 0.05 to 5 μm. In the present invention, the fiber thickness can be measured using a microscope (an electron microscope, an optical microscope, a probe and the like).

The fibrous structure (fibrous material) of the present invention can be entangled with and bind to powder due to its fibrous structure. For example, when tablets are prepared, they can be formed by mixing the ingredients with oxidized PQQ or reduced PQQ powder. This can reduce the amount of a bonding agent that is required for tablet formation produced from oxidized PQQ or reduced PQQ powder. The amount of the reduced PQQ is preferably contained at 1% or more of the total number of moles together with the oxidized PQQ in the final formulated product.

A preferable embodiment of the present invention is a gel comprising a reduced pyrroloquinoline quinone sodium salt prepared by mixing a solution of an oxidized pyrroloquinoline quinone sodium salt and a reducing agent at 50° C. or lower, and a method for producing the same. Here, the weight concentration of the oxidized pyrroloquinoline quinone sodium salt in the mixture (solution) is preferably 0.05% by weight or more and less than 0.7% by weight, more preferably 0.05 to 0.4% by weight, and further preferably 0.05 to 0.3% by weight.

A more preferable embodiment of the present invention is a gel comprising a reduced pyrroloquinoline quinone sodium salt prepared by mixing a solution of an oxidized pyrroloquinoline quinone sodium salt and ascorbic acid solution at 50° C. or lower, and a method for producing the same. Here, the weight concentration of the oxidized pyrroloquinoline quinone sodium salt in the mixture (solution) is preferably 0.05% by weight or more and less than 0.7% by weight, more preferably 0.05 to 0.4% by weight, and further preferably 0.05 to 0.3% by weight.

According to the present invention, the following inventions are also provided.

(1) A gel comprising reduced pyrroloquinoline quinone or a salt thereof represented by Formula (1) and a dispersant,

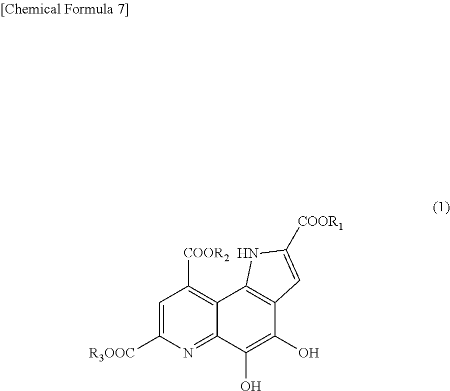

wherein R₁, R₂ and R₃, which may be the same or different, represent a hydrogen atom, a phenyl group, or an alkyl group having 1 to 6 carbon atoms, an aralkyl group, an alkylaryl group, an alkenyl group or an alkynyl group.

(2) The gel according to (1) characterized in that the reduced pyrroloquinoline quinone or a salt thereof is formed into a fibrous structure by physically cross-linking with each other.
(3) The gel according to (1) or (2), wherein the amount of the reduced pyrroloquinoline quinone or a salt thereof is 0.001 to 70% by weight with respect to the total amount of the gel.
(4) The gel according to (3), further including a reducing agent.
(5) The gel according to (3) or (4), wherein the dispersant is water.
(6) The gel according to (3) to (5), further containing an oxidized pyrroloquinoline quinone or a salt thereof represented by Formula (2),

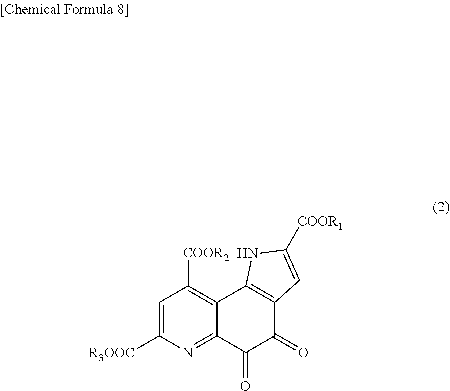

wherein R₁, R₂ and R₃, which may be the same or different, represent a hydrogen atom, a phenyl group, or an alkyl group having 1 to 6 carbon atoms, an aralkyl group, an alkylaryl group, an alkenyl group or an alkynyl group.

(7) The gel according to (6), wherein reduced pyrroloquinoline quinone is contained at 0.1 mol % or more of the total number of moles of pyrroloquinoline quinone.
(8) The gel according to any one of (3) to (7) further containing a macromolecular gellant.
(9) A dry product which is obtained by drying the gel according to any one of (3) to (8).
(10) A film that is made from the gel according to any one of (1) to (8).
(11) A food comprising the gel according to any one of (3) to (8) or the dry product according to (9).
(12) A medicine comprising the gel according to any one of (3) to (8) or the dry product according to (9).
(13) A cosmetic product comprising the gel according to any one of (3) to (8) or the dry product according to (9).
(14) A method for producing a gel, comprising mixing an oxidized pyrroloquinoline quinone or a salt thereof represented by Formula (2):

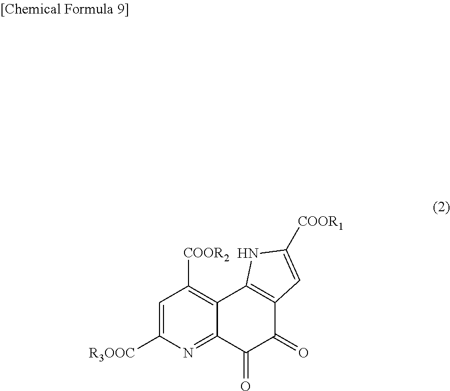

wherein R₁, R₂ and R₃, which may be the same or different, represent a hydrogen atom, a phenyl group, or an alkyl group having 1 to 6 carbon atoms, an aralkyl group, an alkylaryl group, an alkenyl group or an alkynyl group,

and a reducing agent in a dispersant at 50° C. or lower.
(15) The production method according to (14), wherein mixing is performed such that the concentration of pyrroloquinoline quinone in the dispersant is adjusted to 0.05% by weight or more and less than 0.7% by weight.

### EXAMPLES

The present invention will now be described more specifically with reference to the following examples and comparative examples, but is not intended to be limited thereto.

In the present Examples and Comparative Examples, the measurement of diffraction angle 2θ was performed by X-ray powder diffraction (hereinafter, expressed as XRD) under the following measurement conditions.

Equipment: RINT2500 manufactured by RIGAKU CORPORATION
X-ray: Cu/tube voltage of 40 kV/tube current of 100 mA
Scan rate: 4.000°/min
Sampling width: 0.020°

In Examples and Comparative Examples, optical micrographs were taken using a microscope TE-2000S manufactured by NIKON equipped with a ×40 objective lens.

In Examples and Comparative Examples, UV measurements were performed using a HITACHI U-2000 Spectrophotometer.

Oxidized PQQ disodium powder manufactured by Mitsubishi-Gas Chemical Company, Inc. was used as a raw material.

### Example 1

### Preparation of a Gel (1)

Three g of oxidized PQQ disodium powder was dissolved in 1 L of water to prepare a solution containing 3 g/L of PQQ disodium. Ten g of ascorbic acid was dissolved in 90 mL of water to prepare a 10% ascorbic acid solution.

A 4 mL aliquot of oxidized PQQ disodium solution (3 g/L) and a 0.1 mL aliquot of a 10% ascorbic acid solution were placed in a polystyrene test tube, and the resultant mixture was kept at room temperature overnight to obtain a black gel material. The pH was 3.7 during mixing.

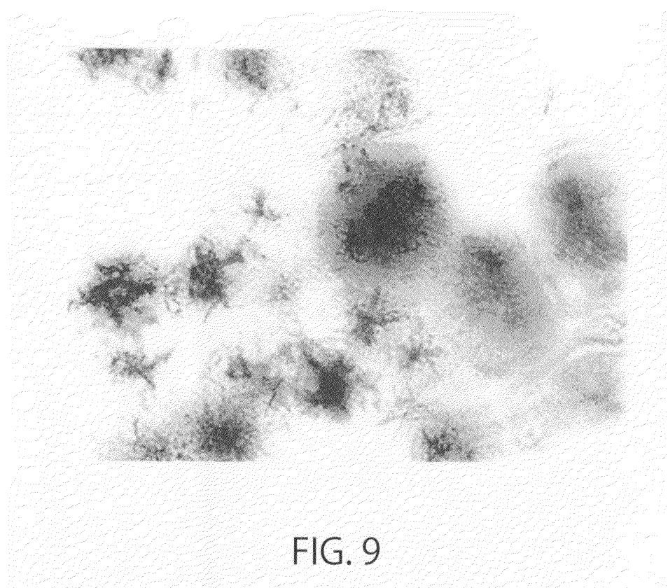

Moreover, this gel was washed with methyl α-hydroxyisobutyrate and observed with an optical microscope. The result is shown in FIG. 9. Crystal formation was not observed, but fibrous form was found.

### Example 2

### Preparation of a Gel (2)

A 4 mL aliquot of oxidized PQQ disodium solution (3 g/L) prepared in the same manner as Example 1 and a 0.2 mL aliquot of 10% ascorbic acid solution were placed in a polystyrene test tube, and the resultant mixture was cooled on ice for 30 minutes. Subsequently, the mixture was kept at room temperature overnight. As a result, a red gel material was obtained. At this time (during mixing), the pH was 3.7.

### Example 3

### Preparation of a Gel (3)

A 4 mL aliquot of oxidized PQQ disodium solution (3 g/L) prepared in the same manner as Example 1 and a 0.1 mL aliquot of 10% ascorbic acid solution were placed in a polystyrene test tube, and the resultant mixture was cooled on ice for 30 minutes. Subsequently the mixture was left in a refrigerator at 10° C. overnight. As a result, a red gel material was obtained. At this time (during mixing), the pH was 3.7.

### Example 4

### Preparation of a Gel (4)

A 4 mL aliquot of oxidized PQQ disodium solution (3 g/L) prepared in the same manner as Example 1 and a 1 mL aliquot of 10% ascorbic acid solution were placed in a polystyrene test tube, and the resultant mixture was cooled on ice for 30 minutes. Subsequently, the mixture was kept in a refrigerator at 10° C. overnight. As a result, a brown gel material was obtained. At this time (while mixing), the pH was 3.1.

### Example 5

### Preparation of a Gel (5)

A 5 g aliquot of oxidized PQQ disodium powder was added to 1 L of water, and the resultant mixture was dissolved at 75° C. This solution was kept at room temperature to make a supersaturated 5 g/L PQQ disodium solution.

A 4 mL aliquot of PQQ disodium solution (5 g/L) and a 1 mL aliquot of 10% ascorbic acid solution were placed in a polypropylene container, and the mixture was cooled in a refrigerator at 10° C. for 1 hour. Subsequently, the mixture was kept at 30° C. for 2 hours, resulting in a dark red gel, and the whole was solidified. At this time (during mixing), the pH was 3.3.

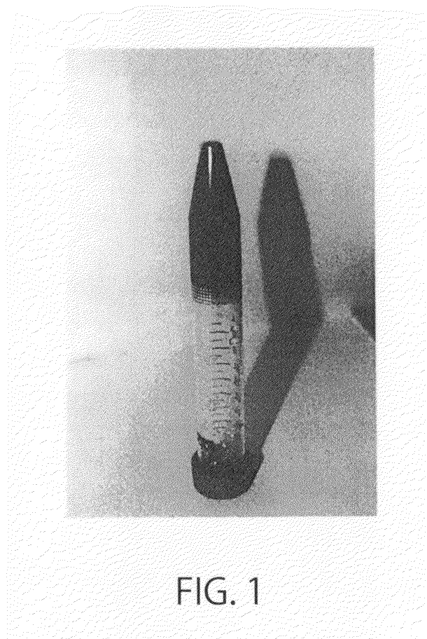

The photograph of the this gel prepared in a 15 mL tube for centrifugation is shown in FIG. 1. The mixture is completely gelated without dripping even though the mixture was turned upside down.

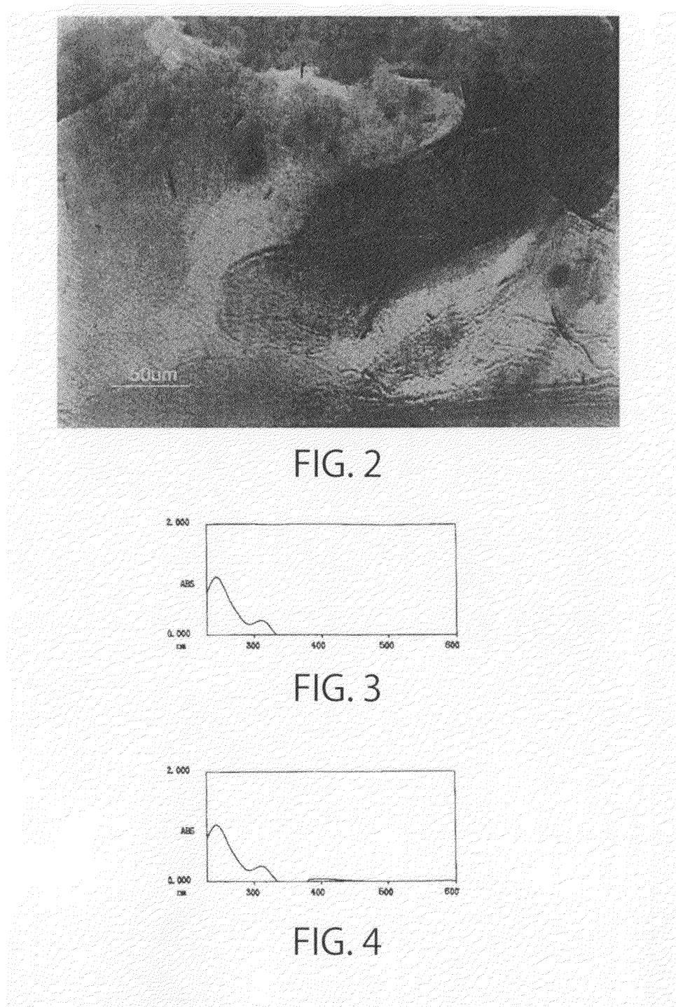

In addition, this gel was washed with methyl α-hydroxyisobutyrate, and the result obtained by observation under an optical microscope is shown in FIG. 2. The gel was observed as a red lump. Moreover, a fibrous material was observed in a part of the gel. Since most gel is formed as fine fiber, the whole gel was seen as a uniform film by observation under an optical microscope.

### Example 6

### Absorbance Measurement by UV Spectrum

Each sample was prepared as follows, and absorbance was measured by using a quartz cell having an optical path length of 1 mm.

### Sample 1: Gel

A 0.20 mL aliquot of oxidized PQQ disodium solution (5 g/L) prepared in the same manner as Example 5 and a 0.05 mL aliquot of 10% ascorbic acid solution were placed in a polypropylene container, and the resultant mixture was cooled in a refrigerator at about 10° C. for 1 hour. Subsequently, the mixture was kept at 30° C. for 2 hours, and the mixture turned to a dark red gel and the whole was solidified. To this, 4.75 mL of water was added. This solution was further diluted with water by a factor of 4. The result of the absorbance measurement is shown in FIG. 3.

Sample 2: A Case of Mixing with Ascorbic Acid (0.05 g/L of PQQ Disodium was Used)

A 0.20 mL aliquot of oxidized PQQ disodium solution (5 g/L) prepared in the same manner as Example 5 and a 0.05 mL aliquot of 10% ascorbic acid solution were placed in a polypropylene container, and immediately after that 4.75 mL of water was added. This solution was further diluted with water by a factor of 4. The result of the absorbance measurement is shown in FIG. 4.

### Sample 3: Reduced PQQ (PQQ Disodium at 0.05 g/L was Used)

A 0.20 mL aliquot of oxidized PQQ disodium solution (5 g/L) prepared in the same manner as Example 5 and a 0.05 mL aliquot of 10% ascorbic acid solution were placed in a polypropylene container, and the resultant mixture was kept at 70° C. for 3 hours, yielding a black solid precipitate.

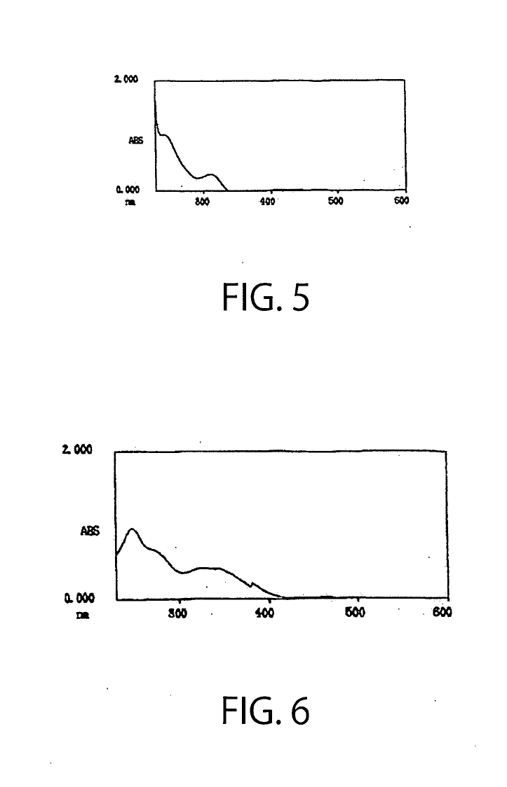

This solid was dissolved by adding 1 mL of dimethyl sulfoxide, and 3.75 ml of water was added. This solution was further diluted with water by a factor of 4. The result of the absorbance measurement is shown in FIG. 5. Note that, since the resultant solid obtained in this experiment was not thoroughly dissolved in water only, this solid was diluted with water after dissolving by using dimethyl sulfoxide, and then the absorbance of this solid was measured.

In addition, hydrochloric acid was added to this solid to adjust the pH to 1 or less. This was centrifuged and the supernatant was discarded. The resultant solid was washed with a degassed aqueous solution of hydrochloric acid, and dried in a nitrogen gas stream. Deuterodimethyl sulfoxide was added to this and the mixture was packed into an NMR tube in a nitrogen gas stream.

By using the JNM-ECA500 500 MHz NMR spectrometer manufactured by JEOL, the 13C-NMR spectrum of the solid was measured at room temperature. As a result, the NMR spectrum was observed at 105.7, 111.0, 119.4, 122.9, 123.6, 128.1, 131.3, 134.2, 137.8, 140.9, 142.6, 162.2, 165.5, and 170.1 ppm (DMSO-d 6: reference at 39.5 ppm). These values were consistent with those of reduced PQQ described in Non Patent Document 5, and the formation of the reduced form was confirmed.

### Sample 4: PQQ Disodium (PQQ Disodium at 0.2 g/L was Used)

A 0.20 mL aliquot of oxidized PQQ disodium solution (5 g/L) prepared in the same manner as Example 5 and water (0.05 mL) were placed in a polypropylene container, and immediately after that 4.75 mL of water was added. The result of the absorbance measurement is shown in FIG. 6.

A large absorbance of the Sample 3 at the wavelength shorter than 240 nm indicates the absorbance of dimethyl sulfoxide used. The absorbance of PQQ disodium changed immediately after mixing with ascorbic acid, and the absorbance at 240 nm increased about 4 times. The absorbance did not change even when the configuration changed to a reduced form of the gel. From these results, it is found that the reduction reaction proceeds simultaneously with the mixing to form the reduced form. Precipitation occurs over the time and the fibrous precipitation leads to gelation.

The amount of the reduced form was calculated when Sample 3 was set as 100% and Sample 4 was set as 0%.

Sample 1 (gel): 100%
Sample 2 (immediately after mixing): 99.8%

### Example 7

### Concentration Procedure of the Gel and Exchange of the Dispersant

The gel prepared in Example 5 was treated with centrifugation at 2000 rpm for 30 minutes. The volume of the gel was reduced to ¼ of the original, and a concentrate was obtained by discarding the supernatant. As the dispersant, 2 N hydrochloric acid, ethanol, isopropanol, and methyl α-hydroxyisobutyrate were used. The dispersant at the same volume of the gel was added, and the supernatant was discarded after centrifugation. This procedure was repeated twice, and the gel with an exchanged dispersant was obtained. The exchange of the dispersant was successfully carried out, and the gel substance was not disintegrated with dissolving. Four kinds of the gel having different dispersants were produced.

### Example 8

### X-Ray Powder Diffraction

The gel obtained in Example 5 was washed with methyl α-hydroxyisobutyrate followed by drying under reduced pressure. The measurement of this sample by X-ray powder diffraction failed and no peaks were observed. The sample was found amorphous.

The gel obtained in the same manner as Example 5 was subjected to filtration treatment at room temperature over the time of 2 hours followed by drying under reduced pressure. A crystallized material represented by the peaks at 8.14, 10.41, 19.74, and 29.94±0.08° was obtained by the measurement of this sample by powder X-ray diffraction. The result of the measurement by X-ray powder diffraction is shown in FIG. 7.

### Comparative Example 1

A 4 mL aliquot of PQQ oxidized disodium solution (5 g/L) prepared in the same manner as Example 5 and a 1 mL aliquot of 10% ascorbic acid solution were placed in a polystyrene test tube, and the resultant mixture was cooled in a refrigerator at about 10° C. for 1 hour. After that, maintaining this at 70° C. for 2 hours resulted in precipitation of a black solid. The reduced PQQ powder was obtained. As a result, this solid was found amorphous by X-ray powder diffraction analysis.

### Comparative Example 2

A 4 mL aliquot of oxidized PQQ disodium solution (5 g/L) prepared in the same manner as Example 5 was cooled on ice for 1 hour and then kept at 30° C. for 2 hours. Although crystals were precipitated, gel formation was not observed.

### Example 9

### Mixing of the Gel and Oxidized PQQ

One mL of the gel prepared in the same manner as Example 5 and 0.3 g of PQQ disodium powder were mixed. A uniform mixture was obtained. The amount of the reduced product in the mixture was 1.6%. This was dried under reduced pressure, and a solidified red solid was obtained.

### Example 10

### Preparation of a Fibrous Material in Agar (Agarose)

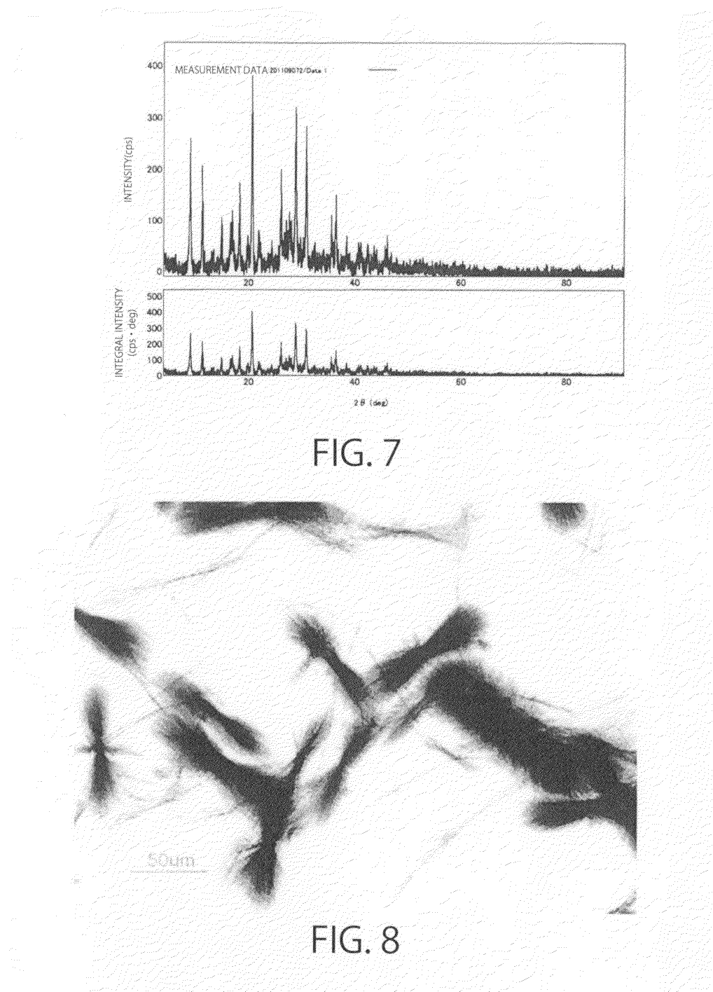

A 0.40 g aliquot of Agarose from Wako Pure Chemical Industries, Ltd. was added to a 40 mL aliquot of oxidized PQQ disodium solution (3 g/L) prepared in the same manner as. Example 1. The resultant mixture was heated in a microwave oven to completely dissolve Agarose. Four mL of this solution was placed in a petri dish, and the solution was cooled and solidified at room temperature. To this, 1 mL of 10% of ascorbic acid was added, and the resultant mixture was kept on ice for 1 hour. Subsequently, the temperature of the solution was kept at 30° C. for 1 hour. Formation of a fibrous material was observed under an optical microscope. The microscopic image is shown in FIG. 8. The fibrous material still remained after further keeping the sample overnight. Moreover, a fibrous material was generated in agar.

### Example 11

### Gelation in the Presence of a Sugar

To each of 4 mL aliquots of oxidized PQQ disodium solution (5 g/L) prepared in the same manner as Example 5, 0.3, 0.6, 0.9, 1.2, and 1.5 g of sorbitol was added, and the each resultant mixture was dissolved. To each of these mixtures, a 1 mL aliquot of 10% ascorbic acid solution was added, and the mixtures were cooled in a refrigerator at about 10° C. for 1 hour. Subsequently, when the mixtures were kept at 30° C. for 2 hours, all the mixtures turned to a dark red gel and the whole was solidified. However, the mixture to which 1.2 or 1.5 g of sorbitol was added generated a soft gel. The addition of a sugar makes gel formation difficult, however, gel formation was shown to proceed even at a sugar concentration of 20% by weight or more, and it had practically no problem.

### Example 12

### Preparation of a Film from the Gel

One mL of the gel in Example 5 was added to 10 mL of methanol, and the resultant mixture was centrifuged. After discarding the supernatant, 1 mL of methanol was added to the residue and placed in a petri dish. After drying under reduced pressure, a film was obtained on the petri dish.

### Example 13

### Preparation of a Film from the Gel

One mL of the gel in Example 5 was added to 10 mL of 2-propanol, and the resultant mixture was centrifuged. After discarding the supernatant, 1 mL of 2-propanol was added to the residue and placed in a petri dish. After drying under reduced pressure, a film was obtained on the petri dish.

### Example 14

### Preparation of a Film from the Gel

Four mL of the gel in Example 5 was placed on a filter No. 5, and the gel was filtrated under suction. The residue was washed with 20 mL of 2-propanol, and dried along with the filter. As a result, the filter was uniformly coated with the gel.

### Comparative Example 3

The mixture of Comparative Example 1 obtained by reacting at 75° C. was centrifuged, and the supernatant was discarded. Subsequently, this residue was washed with 10 mL of 2-propanol, and 1 mL of 2-propanol was added to this, and the resultant mixture was placed in a petri dish followed by drying under reduced pressure. However, crystals were scattered on the petri dish and a film was not observed.

### Comparative Example 4

A 4 mL aliquot of oxidized PQQ disodium solution (5 g/L) prepared in the same manner as Example 5 and a 1 mL aliquot of 10% ascorbic acid solution were mixed, and the resultant mixture was immediately dried under reduced pressure. Because drying was started immediately after the mixing, water was evaporated before gelation. Therefore, powders were found sparsely and a film was not observed.

### Example 15 to 17 and Comparative Example 5 to 6

The raw material that was the same as in Example 1 was used, and experiments were performed by changing the volume of 10% ascorbic acid solution and the temperature. The results are shown as follows.

TABLE 1

| | 3g/L of | 10% | | | |
|---|---|---|---|---|---|
| | PQQ | ascorbic | | | After |
| | disodium | acid | | | one |
| | solution | solution | | Temperature | day |
| Example 1 | 4 mL | 100 | μL | 25° C. | gelation |
| Example 15 | 4 mL | 500 | μL | 25° C. | gelation |
| Example 16 | 4 mL | 50 | μL | 25° C. | gelation |
| Example 17 | 4 mL | 20 | μL | 25° C. | gelation |
| Comparative | 4 mL | 100 | μL | 70° C. | Precipitation |

| | | | | |
|---|---|---|---|---|
| Example 5 | | | | of |
| | | | | black |
| | | | | reduced-form |
| | | | | crystals |
| Comparative | 4 mL | 0 | 25° C. | solution |
| Example 6 | | | | |

Gelation was not observed when the reaction was carried out at 70° C. Moreover, it was confirmed that the amount of ascorbic acid added did not affect gelation.

### INDUSTRIAL APPLICABILITY

A gel of the present invention is advantageous in the fields of food, functional foods, pharmaceutical agents, quasi-drugs, functional materials or cosmetics and the like.

1. A gel comprising a reduced pyrroloquinoline quinone derivative represented by Formula (1) or a salt thereof, and a dispersant: 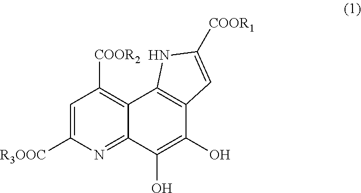
wherein R₁, R₂ and R₃ each independently represent a hydrogen atom, a phenyl group, an alkyl group having 1 to 6 carbon atoms, an aralkyl group, an alkylaryl group, an alkenyl group or an alkynyl group.

2. The gel according to claim 1, wherein molecules of the reduced pyrroloquinoline quinone derivative or salt thereof are associated with each other to form a fibrous structure.

3. The gel according to claim 1, wherein the reduced pyrroloquinoline quinone derivative or salt thereof is present in an amount of 0.001 to 70% by weight based on a total weight of the gel.

4. The gel according to claim 1, wherein the reduced pyrroloquinoline quinone derivative or salt thereof is present in an amount of 0.05% by weight or more and less than 0.7% by weight based on a total weight of the gel.

5. The gel according to claim 1, further comprising a reducing agent.

6. The gel according to claim 1, wherein the dispersant is water.

7. The gel according to claim 1, further comprising an oxidized pyrroloquinoline quinone derivative or a salt thereof represented by Formula (2): 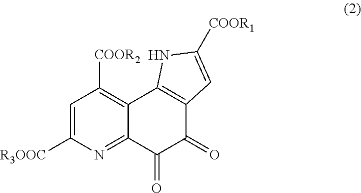
wherein R₁, R₂ and R₃ each independently represent a hydrogen atom, a phenyl group, an alkyl group having 1 to 6 carbon atoms, an aralkyl group, an alkylaryl group, an alkenyl group or an alkynyl group.

8. The gel according to claim 7, wherein the reduced pyrroloquinoline quinone derivative or salt thereof is present in an amount of 0.1 mol % or more based on a total molar amount of the reduced pyrroloquinoline quinone derivative or salt thereof and the oxidized pyrroloquinoline quinone derivative or salt thereof.

9. The gel according to claim 1, further comprising a macromolecular gellant.

10. A dry product obtained by drying the gel according to claim 1.

11. A film produced from the gel according to claim 1.

12. A food product comprising the gel according to claim 1.

13. A pharmaceutical product comprising the gel according to claim 1.

14. A cosmetic product comprising the gel according to claim 1.

15. A method for producing a gel comprising a reduced pyrroloquinoline quinone derivative represented by Formula (1) or a salt thereof: 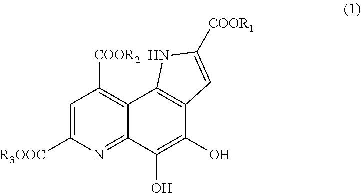
wherein R₁, R₂ and R₃ each independently represent a hydrogen atom, a phenyl group, an alkyl group having 1 to 6 carbon atoms, an aralkyl group, an alkylaryl group, an alkenyl group or an alkynyl group,
the method comprising mixing an oxidized pyrroloquinoline quinone derivative represented by Formula (2) or a salt thereof: 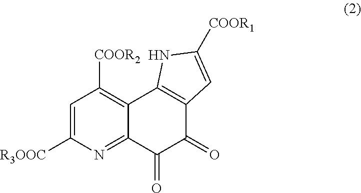
wherein R₁, R₂ and R₃ each independently represent a hydrogen atom, a phenyl group, an alkyl group having 1 to 6 carbon atoms, an aralkyl group, an alkylaryl group, an alkenyl group or an alkynyl group,
with a dispersant and a reducing agent at 50° C. or lower.

16. The method according to claim 15, wherein a concentration of the oxidized pyrroloquinoline quinone derivative or salt thereof in the dispersant is 0.05% by weight or more and less than 0.7% by weight.
